(19) United States
(12) Patent Application Publication
(10) Pub. No.: US 2026/0278748 A1
(43) Pub. Date: Sep. 17, 2026

(54) MULTIFREQUENCY ULTRASOUND SYSTEMS AND METHODS

(71) Applicant: The Texas A&M University System, College Station, TX (US)

(72) Inventors: Kenneth Hoyt, Bryan, TX (US); Joshua Hanson, Cypress, TX (US)

(21) Appl. No.: 19/557,977

(22) Filed: Mar. 5, 2026

Related U.S. Application Data
(60) Provisional application No. 63/770,845, filed on Mar. 12, 2025.

(51) Int. Cl. G06T 5/70; A61B 8/00

(52) U.S. Cl. CPC G06T 5/70; A61B 8/5207; A61B 8/5269; G06T 2207/10132

(57) ABSTRACT

Disclosed are systems and methods for improved ultrasound imaging and analysis of ultrasound data. A system includes an ultrasound transducer configured to iteratively transmit an ultrasound signal into a specimen at a plurality of frequencies, detect backscattered ultrasound at each of the plurality of frequencies, and generate radiofrequency (RF) data for each of the plurality of frequencies. A postprocessing unit receives at least a portion of the RF data and transforms the data into a raw image comprising first pixels, each comprising an intensity value. For each pixel of the raw image, an attenuation coefficient is determined. Attenuation-corrected RF data is generated using the determined attenuation coefficients. The attenuation-corrected RF data is transformed into an attenuation-corrected ultrasound image, which is provided for display on a display device.

### CROSS-REFERENCE TO RELATED APPLICATIONS

This application claims the priority of U.S. Provisional Appl. Ser. No. 63/770,845, filed Mar. 12, 2025, the entire contents of which are incorporated herein by reference.

### STATEMENT OF GOVERNMENT RIGHTS

This invention was made with government support under R01CA269973 awarded by the National Institutes of Health and the National Cancer Institute. The government has certain rights in the invention.

### FIELD OF TECHNOLOGY

The present invention relates generally to the field of ultrasound imaging. More specifically, the invention relates, in certain embodiments, to multifrequency ultrasound systems and methods.

### BACKGROUND

Ultrasound techniques are used for tissue imaging and characterization. As an ultrasound signal propagates through a material like soft tissue, there is a progressive decay in signal amplitude. During this propagation, higher frequency signals become more attenuated than lower frequency signals, resulting in a pronounced spectral shift in backscattered ultrasound signal content. Attenuation is measured by an ultrasound attenuation coefficient. The ultrasound attenuation coefficient measures the gradual loss of ultrasound signal amplitude as it propagates to greater depths in a sample, such as a tissue. Causes of ultrasound attenuation include absorption, reflection, scattering, and refraction. Ultrasound attenuation is dependent on the frequency of the ultrasound used during imaging (higher frequency waves experience greater attenuation), tissue type (different tissues have varying attenuation coefficients), and tissue depth (attenuation increases with the distance traveled by the ultrasound waves).

While ultrasound attenuation has a negative impact on traditional ultrasound image quality, the ultrasound attenuation coefficient can be useful for tissue characterization. Quantitative ultrasound techniques have been developed that use attenuation measures for tissue characterization. For example, quantitative ultrasound techniques have been developed which use estimates of the ultrasound attenuation coefficient to quantify fat content in the liver and monitor progression of metabolic dysfunction-associated fatty liver disease (MAFLD). Information derived from quantitative ultrasound can reduce subjectivity in clinical diagnostics by offering non-invasive insight into tissue composition and pathology (K. Nam, et al., Ultrason. Imaging, vol. 33, no. 4, pp. 251-263, 2011). For example, quantitative ultrasound attenuation measures have been used for the differentiation of normal and steatotic liver disease as well as for distinguishing between benign and malignant tumors (J. Baek et al., Med. Phys., vol. 51, no. 2, pp. 1313-1325, 2024; L. Basavarajappa et al., Sci. Rep., vol. 11, no. 1, p. 2655, 2021; B. W. Dong, et al., J. Clin. Ultrasound, vol. 22, no. 3, pp. 167-174, 1994). Other potential clinical applications of quantitative ultrasound include bone disease evaluation and assessment of cervical condition during pregnancy (Y. Karbalaeisadegh and M. Muller, Adv. Exp. Med. Biol., vol. 1364, pp. 177-196, 2022; B. L. McFarlin, et al., Ultrasound Obstet. Gynecol., vol. 36, no. 2, pp. 218-225, 2010). In the field of interventional medicine, ultrasound attenuation measurements have helped in monitoring thermal lesions during ablation therapy (M. D. Gray, et al., Med. Phys., vol. 51, no. 2, pp. 809-825, 2024). As research continues to expand the applications of quantitative ultrasound, attenuation measurements are likely to become an increasingly important component of clinical examinations across various medical specialties.

The two most commonly used attenuation coefficient estimation methods include the spectral shift method and the reference phantom method. The spectral shift method ignores ultrasound system effects, such as beamforming and diffraction effects, thereby limiting estimation accuracy. This limitation arises because the spectral shift method assumes that changes in the frequency spectrum of backscattered ultrasound are caused solely by tissue attenuation, when in fact ultrasound system characteristics also affect the spectrum. The reference phantom method requires access to a well-calibrated phantom, which is not available in many scenarios. Furthermore, phantom properties can change over time as the material degrades, leading to drift in calibration accuracy. Both conventional methods also typically estimate only a single global attenuation coefficient for an entire region-of-interest, which assumes tissue homogeneity. This assumption is often incorrect for real biological samples, such as heterogeneous tissues containing multiple tissue types or pathological regions, leading to inaccurate attenuation estimates and suboptimal image correction. Thus, there remains a need for improved tools for estimating ultrasound attenuation coefficient values that do not require a reference phantom while still being independent of ultrasound system effects.

### SUMMARY

This disclosure not only encompasses the recognition of the problems of previous ultrasound imaging technologies, such as those described above, but also provides solutions to these and other technological problems. For instance, the systems and methods of this disclosure provide improvements to both qualitative ultrasound imaging and quantitative ultrasound measurements. For example, this disclosure recognizes that quantitative ultrasound can provide complementary information to that obtained from qualitative ultrasound imaging and that improvements to ultrasound attenuation measures for quantitative ultrasound can also be used to improve qualitative ultrasound imaging by providing improved correction of signal attenuation during ultrasound imaging. Previous technology to correct for ultrasound signal attenuation relies on a global attenuation value for a sample or a defined homogeneous region of the sample. Given that this assumption of homogeneity is often incorrect for real samples, such as heterogeneous tissues, an attenuation map-based approach is provided in this disclosure that can both improve the accuracy of ultrasound imaging while also providing improved information for quantitative ultrasound. This information can be combined to provide both improved qualitative ultrasound images and higher resolution quantitative ultrasound data. To facilitate the above improvements, embodiments of this disclosure are directed to an improved ultrasound attenuation coefficient estimation procedure that facilitates spatial mapping using an ultrasound system. This technology can provide improved tissue characterization using multifrequency ultrasound imaging system combined with improved attenuation calculation and correction. Importantly, the systems and methods disclosed herein improve the functioning of the ultrasound imaging system itself by enabling pixel-by-pixel attenuation correction that was not previously achievable with conventional single-frequency or global-attenuation approaches. The disclosed multifrequency transmission and acquisition strategy, combined with the specific algorithmic processing steps, represents an unconventional technological solution that fundamentally changes how attenuation data is acquired and processed in ultrasound systems, resulting in images with improved diagnostic quality and accuracy.

An aspect of the present disclosure provides a system, comprising: an ultrasound transducer configured to: iteratively transmit an ultrasound signal into a specimen at a plurality of frequencies; detect backscattered ultrasound at each of the plurality of frequencies; and generate radiofrequency (RF) data for each of the plurality of frequencies; and a postprocessing unit comprising a processor configured to: receive at least a portion of the RF data; transform the received RF data into a raw image comprising first pixels, each first pixel comprising an intensity value (e.g., associated with an intensity of the backscattered ultrasound); for each first pixel of the raw image, determine an attenuation coefficient based on at least one of: the intensity value of the first pixel, a transmission frequency at which the intensity value was obtained, and a depth within the specimen at which the intensity value was obtained; generate attenuation-corrected RF data using the determined attenuation coefficients; transform the attenuation-corrected RF data into an attenuation-corrected ultrasound image comprising second pixels, each second pixel comprising an attenuation-corrected intensity value determined based at least in part on the determined attenuation coefficients; and provide the attenuation-corrected image for display on a display device.

In some embodiments, the processor of the postprocessing unit is further configured to determine the attenuation coefficient based on each of the intensity value of the first pixel, the transmission frequency at which the intensity value was obtained, and the depth within the specimen at which the intensity value was obtained. In some embodiments, the processor of the postprocessing unit is further configured to, for each frequency of the plurality of frequencies, generate the attenuation-corrected RF data using a correction factor with an exponential term that comprises an attenuation coefficient of the determined attenuation coefficients and the frequency. In some embodiments, the processor of the postprocessing unit is further configured to normalize the RF data to compensate for intensity changes of the backscattered ultrasound at the plurality of frequencies of the ultrasound signal transmitted by the ultrasound transducer.

In some embodiments, the processor of the postprocessing unit is further configured to filter the RF data. In some embodiments, the processor of the postprocessing unit is further configured to: generate an attenuation map comprising a plurality of third pixels, each third pixel comprising the attenuation coefficient determined for each first pixel of the raw image; and provide the attenuation map for display on the display device. In some embodiments, the processor of the postprocessing unit is further configured to: determine a maximum attenuation coefficient and a minimum attenuation coefficient; and color code the attenuation map on a scale based on the maximum attenuation coefficient and the minimum attenuation coefficient (e.g., or based on a user-defined range). In some embodiments, the processor of the postprocessing unit is further configured to receive a broadband ultrasound image of the specimen (e.g., with the same field of view used to determine the attenuation coefficients) and adjust the broadband ultrasound image using the attenuation coefficients, thereby generating an enhanced broadband ultrasound image.

Another aspect of the present disclosure provides a method, comprising: iteratively transmitting, by an ultrasound transducer, an ultrasound signal into a specimen at a plurality of frequencies; detecting backscattered ultrasound at each of the plurality of frequencies; generating radiofrequency (RF) data for each of the plurality of frequencies; and transforming at least a portion of the RF data into a raw image comprising first pixels, each first pixel comprising an intensity value; for each first pixel of the raw image, determining an attenuation coefficient based on at least one of: the intensity value of the first pixel, a transmission frequency at which the intensity value was obtained, and a depth within the specimen at which the intensity value was obtained; generating attenuation-corrected RF data using the determined attenuation coefficients; transforming the attenuation-corrected RF data into an attenuation-corrected ultrasound image comprising second pixels, each second pixel comprising an attenuation-corrected intensity value determined based at least in part on the determined attenuation coefficients; and providing the attenuation-corrected image for display on a display device.

In some embodiments, the method further comprises determining the attenuation coefficient based on each of the intensity value of the first pixel, the transmission frequency at which the intensity value was obtained, and the depth within the specimen at which the intensity value was obtained. In some embodiments, the method further comprises, for each frequency of the plurality of frequencies, generating the attenuation-corrected RF data using a correction factor with an exponential term that comprises an attenuation coefficient of the determined attenuation coefficients and the frequency. In some embodiments, the method further comprises normalizing the RF data to compensate for intensity changes of the backscattered ultrasound at the plurality of frequencies of the ultrasound signal transmitted by the ultrasound transducer.

In some embodiments, the method further comprises filtering the RF data. In some embodiments, the method further comprises: generating an attenuation map comprising a plurality of third pixels, each third pixel comprising the attenuation coefficient determined for each first pixel of the raw image; and providing the attenuation map for display on the display device. In some embodiments, the method further comprises: determining a maximum attenuation coefficient and a minimum attenuation coefficient; and color coding the attenuation map on a scale based on the maximum attenuation coefficient and the minimum attenuation coefficient. In some embodiments, the method further comprises receiving a broadband ultrasound image of the specimen (e.g., with the same field of view used to determine the attenuation coefficients) and adjusting the broadband ultrasound image using the attenuation coefficients, thereby generating an enhanced broadband ultrasound image.

Yet another aspect of the present disclosure provides a system, comprising a processor configured to: receive RF data generated by an ultrasound transducer at a plurality of frequencies, the RF data associated with a specimen; transform the received RF data into a raw image comprising first pixels, each first pixel comprising an intensity value; for each first pixel of the raw image, determine an attenuation coefficient based on at least one of: the intensity value of the first pixel, a transmission frequency at which the intensity value was obtained, and a depth within the specimen at which the intensity value was obtained; generate attenuation-corrected RF data using the determined attenuation coefficients; transform the attenuation-corrected RF data into an attenuation-corrected ultrasound image comprising second pixels, each second pixel comprising an attenuation-corrected intensity value determined based at least in part on the determined attenuation coefficients; and provide the attenuation-corrected image for display on a display device.

In some embodiments, the processor is further configured to determine the attenuation coefficient based on each of the intensity value of the first pixel, the transmission frequency at which the intensity value was obtained, and the depth within the specimen at which the intensity value was obtained. In some embodiments, the processor is further configured to, for each frequency of the plurality of frequencies, generate the attenuation-corrected RF data using a correction factor with an exponential term that comprises an attenuation coefficient of the determined attenuation coefficients and the frequency. In some embodiments, the processor is further configured to normalize the RF data to compensate for intensity changes of backscattered ultrasound detected by the ultrasound transducer at the plurality of frequencies of the ultrasound signal transmitted by the transducer.

In some embodiments, the processor is further configured to filter the RF data. In some embodiments, the processor is further configured to: generate an attenuation map comprising a plurality of third pixels, each third pixel comprising the attenuation coefficient determined for each first pixel of the raw image; and provide the attenuation map for display on the display device.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a block diagram of an example ultrasound system, according to embodiments of this disclosure.

FIG. 2 shows a block-flow diagram of an example method of attenuation correction and attenuation map generation, according to embodiments of this disclosure.

FIG. 3 shows a diagram illustrating attenuation coefficient estimation, according to embodiments of this disclosure.

FIG. 4A shows an example raw ultrasound image of an ultrasound phantom specimen.

FIG. 4B shows an example attenuation map corresponding to the image of FIG. 4A.

FIG. 4C shows an example attenuation-corrected ultrasound image corresponding to the image of FIG. 4A.

FIG. 5 shows a box chart of attenuation coefficients determined for an ultrasound phantom specimen.

FIG. 6 shows ultrasound images and RF lines of a specimen before and after attenuation correction as imaged from both the low attenuation (Low ATT) and high attenuation (High ATT) sides of the specimen.

### DETAILED DESCRIPTION

As described above, previous ultrasound imaging suffered from various drawbacks and disadvantages, for example, associated with attenuation limiting ultrasound image quality and limitations imposed by the limited accuracy of localized attenuation estimation in ultrasound data. Embodiments of this disclosure provide solutions to these problems of previous technology, for example, by facilitating improved local ultrasound attenuation estimation using a multifrequency ultrasound imaging approach that is capable of accurate pixel-by-pixel estimation of ultrasound attenuation values, which can be used for both tissue characterization and image correction. Unlike conventional approaches that estimate a single global attenuation value or require calibration phantoms, the disclosed systems and methods leverage a specific, unconventional combination of multifrequency signal acquisition and pixel-wise algorithmic processing to achieve spatially-resolved attenuation mapping. This approach represents a technological advancement that improves the accuracy and diagnostic utility of ultrasound imaging systems.

The disclosed systems integrate both hardware and software in a specific technological configuration to solve problems of previous ultrasound imaging technology. Embodiments of the multifrequency ultrasound imaging system and method involve using the waveform generation capability of a programmable ultrasound system to implement a frequency-shifted narrowband ultrasound transmission pulsing strategy. This hardware-level modification to ultrasound signal transmission, combined with the corresponding software-based processing algorithms, represents an integrated solution that could not be achieved by either hardware or software modifications alone. The multifrequency transmission approach exploits the physical phenomenon that ultrasound attenuation is frequency-dependent, with higher frequencies experiencing greater attenuation. By systematically varying the transmission frequency and analyzing the resulting intensity variations at each spatial location, the system can isolate and quantify the local attenuation properties of the tissue. This approach allows a specimen to be subjected to a series of different frequency ultrasound pulses. The resulting backscattered ultrasound signals are measured, and the data are combined using the disclosed algorithms to generate a high-resolution spatial map of ultrasound attenuation. This map can be used directly for tissue characterization and/or used to correct attenuation in ultrasound images on a pixel-by-pixel basis for further quantitative analysis.

The multifrequency ultrasound imaging systems and methods of this disclosure may facilitate improved tissue characterization techniques. The systems and methods of this disclosure provide information related to changes in backscattered ultrasound signal intensity to be measured as a function of both depth and frequency, which are key parameters to quantitative ultrasound attenuation measurements. In certain embodiments, multiple frequency-shifted narrowband ultrasound pulses are transmitted into a specimen or other sample (e.g., in a region-of-interest), and the resulting backscattered ultrasound is measured and used for improved estimation of attenuation coefficient as a function of position in the specimen or sample. For instance, the systems and methods may iteratively scan at a plurality of frequencies to obtain measurements across a relatively broad frequency range. In an embodiment, a lower frequency narrowband ultrasound pulse is transmitted and backscattered ultrasound data is recorded and processed using a matched filter design. The center frequency of the narrowband ultrasound pulse is then incremented, and the process is repeated until backscattered ultrasound data is obtained throughout the entire transducer bandwidth (or a selected bandwidth of interest for a given application). Data from all frequencies is compiled and used to estimate attenuation coefficients, generate an attenuation map or other attenuation for quantitative analysis, and/or generate attenuation-corrected ultrasound images, as described in greater detail with respect to the examples described below.

### I. Example Multifrequency Ultrasound System

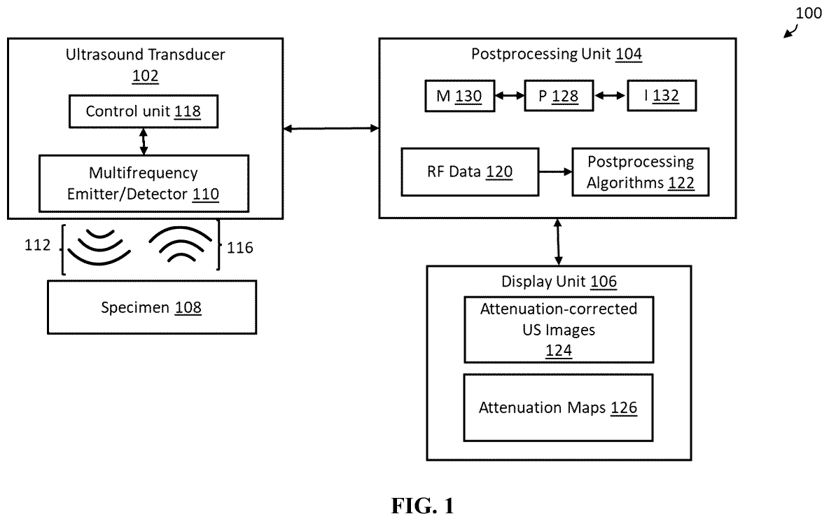

FIG. 1 shows an example ultrasound system 100 of this disclosure. The ultrasound system 100 includes an ultrasound transducer 102, a postprocessing unit 104, and a display unit 106. The ultrasound transducer 102 is used to transmit an ultrasound signal 112 into a specimen 108 and receive a backscattered ultrasound signal 116 (sometimes referred to as “ultrasound echoes”) from the specimen 108. Radiofrequency (RF) data 120 corresponding to the detected backscattered ultrasound signal 116 is provided to the postprocessing unit 104, which employs improved postprocessing algorithms 122 to correct for attenuation of the ultrasound signals 112, 116 by the specimen and generate an attenuation-corrected ultrasound image 124 of the specimen 108 (or a portion of the specimen 108). In some embodiments, an attenuation map 126 is generated that shows the spatial distribution of attenuation coefficient determined in the specimen 108. As described elsewhere in this disclosure, the attenuation-corrected ultrasound image 124 and the attenuation map 126 facilitate improved specimen characterization (e.g., for disease diagnosis, monitoring, etc.).

The ultrasound transducer 102 includes an ultrasound emitter/detector 110 that transmits an ultrasound signal 112 at a plurality of frequencies and detects backscattered ultrasound signal 116 (e.g., ultrasound echoes) at the same frequencies. RF data 120 is generated corresponding to the intensity of the backscattered ultrasound signal 116 and provided to the postprocessing unit 104, described below. The multifrequency ultrasound emitter/detector 110 may include one or more ultrasound emitters, such as piezoelectric crystals or other ultrasound transducer(s), which are each configured to emit ultrasound at a different frequency. In some embodiments, the emitter/detector 110 includes a capacitive micromachined ultrasound transducer (CMUT) able to transmit ultrasound with programmable frequency characteristics (e.g., with a programmable waveform of the ultrasound signal 112). Use of a CMUT offers a broader bandwidth and higher spatial resolution compared to standard piezoelectric transducers. For tissue characterization, a broader frequency range provides more information on frequency-dependent ultrasound backscatter leading to improved sensitivity and more accurate scatterer size estimates. The emitter/detector 110 may be programmable and capable of emitting ultrasound signal 112 with different programmed waveforms. In some embodiments, the emitter/detector 110 is configured to emit ultrasound signal 112 as a number of ultrasound pulses. In some embodiments, a custom multifrequency-shifted narrowband transmit pulsing is employed to allow complete use of the broader bandwidth available to a CMUT and further improve correction of local attenuation in ultrasound images.

The example ultrasound transducer 102 of FIG. 1 includes a control unit 118 that is communicatively coupled to the emitter/detector 110. The control unit 118 controls operation of the emitter/detector 110 and allows communication of data (e.g., RF data 120) to the postprocessing unit 104. For example, the control unit 118 may include control circuitry, such as a controller with a processor, memory, and communications interface, which causes the emitter/detector 110 to emit ultrasound signal 112 and generate RF data 120 corresponding to detected backscattered ultrasound signal 116. As an example, the control unit 118 may cause the ultrasound emitter/detector 110 to transmit the ultrasound signal 112 at a plurality of angles into the specimen 108, detect backscattered ultrasound signal 116, generate an electronic signal (e.g., RF data 120) corresponding to the intensity of the backscattered ultrasound signal 116, optionally preprocess the RF data 120, and provide the raw or preprocessed RF data 120 for use by the postprocessing unit 104. Further details and examples of the operation of an ultrasound transducer 102 are provided below.

The postprocessing unit 104 receives at least a portion of the RF data 120 generated by the ultrasound transducer 102 and transforms the RF data 120 into an attenuation-corrected ultrasound image 124 (e.g., based on a broadband ultrasound image included in the RF data 120) and/or an attenuation map 126 using the improved postprocessing algorithm 122. Briefly, the RF data 120 may initially be adjusted. For example, if the RF data 120 is initially received in a beamformed in-phase and quadrature (IQ) format, the RF data 120 may be converted to a radiofrequency format for subsequent processing. The RF data 120 may be normalized to compensate for intensity changes of the backscattered ultrasound signal 116 at the different frequencies of the ultrasound emitter/detector 110. The RF data 120 may be filtered (e.g., to remove signals with frequencies that do not correspond to the transmission frequencies of the transmitted ultrasound signal 112).

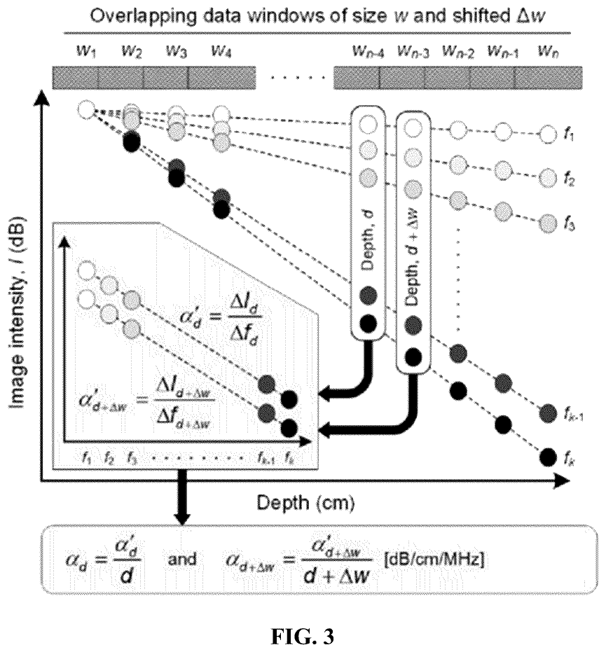

The post-processing algorithm 122 is then used to transform (e.g., convert, translate) the RF data 120 (whether as received or after normalization and/or other processing steps) into raw ultrasound image data. Each pixel of the ultrasound image data has an intensity value, which corresponds to an intensity of the backscattered ultrasound signal 116 from the region in the specimen 108 corresponding to the pixel location in the raw image data. For each pixel (or optionally a subset of the pixels) of the raw image data, an attenuation coefficient is determined based on the intensity value of the pixel, a transmission frequency at which the intensity value was obtained, and/or a depth within the specimen 108 at which the intensity value was obtained. Each of these features may be used to determine the attenuation coefficients (see FIG. 3 and corresponding description below for more details on determining attenuation coefficients). As an example, to determine the attenuation coefficients, a plurality of longer narrowband ultrasound pulses are transmitted into the specimen 108.

Attenuation-corrected RF data is then generated using the determined attenuation coefficients. For example, conventional broadband ultrasound images may be obtained after the attenuation coefficients are determined, as described above (e.g., using longer narrowband pulses at a number of frequencies). The shorter pulses of broadband ultrasound imaging have a higher axial resolution, which is beneficial for diagnostic imaging. Attenuation-corrected RF data may then be determined by applying a correction factor with an exponential term that has the attenuation coefficient associated with the pixel coordinate to the broadband ultrasound image for the data range and the frequency at which the data was collected (see Equation 1 below). The attenuation-corrected RF data is then transformed (e.g., converted, translated) into the attenuation-corrected ultrasound image 124. Each pixel of the attenuation-corrected ultrasound image 124 includes an attenuation-corrected intensity value determined based at least in part on the attenuation coefficients. The attenuation-corrected ultrasound image 124 is provided for display on the display unit 106. The display unit 106 includes at least one image display device, such as an electronic display, a computer monitor, a touchscreen device, or the like.

The postprocessing unit 104 may generate an attenuation map 126 that can be used to visualize the spatial distribution of attenuation coefficients in the specimen 108. The attenuation map 126 includes a plurality of third pixels, each including the attenuation coefficient determined for the corresponding pixel of the raw image data. The attenuation map 126 is provided for display on the display unit 106. The attenuation map 126 may be color coded (e.g., using grayscale, a heat map, or another color and/or intensity scheme) to allow visualization of the distribution of attenuation coefficient in the specimen 108. For example, the postprocessing unit 104 may determine a maximum attenuation coefficient and a minimum attenuation coefficient and color code the attenuation map 126 on a scale based on the maximum attenuation coefficient and the minimum attenuation coefficient (e.g., such that the color scale extends between the maximum and minimum values).

The postprocessing unit 104 includes a processor 128, memory 130, and communications interface 132 (e.g., to communicate with the transducer 102 and display unit 106). The processor 128 executes instructions stored in memory 130 to perform the operations described in this disclosure. The processor 128 may include one or more processors. The memory 130 stores RF data 120, attenuation-corrected ultrasound images 124, and attenuation maps 126 along with any other information generated to obtain this data. The memory 130 also stores instructions, such as any logic and/or code for implementing the postprocessing algorithms 122. The memory 130 may include one or more disks, tape drives, or solid-state drives, and may be used as an over-flow data storage device, to store programs when such programs are selected for execution, and to store instructions and data that are read during program execution. The memory 130 may be volatile or non-volatile and may comprise read-only memory (ROM), random-access memory (RAM), ternary content-addressable memory (TCAM), dynamic random-access memory (DRAM), and/or static random-access memory (SRAM). The communications interface 132 couples the postprocessing unit 104 to the ultrasound transducer 102 and the display unit 106. The communications interface 132 may include any appropriate communication interface(s), including, but not limited to, universal serial bus (USB) port(s), other serial port(s) (e.g., RS232 port(s), ethernet port(s), other networking port(s) (e.g., WAN and LAN port(s)), controller area network (CAN) bus port(s), and the like. The communications interface 132 may use wired and/or wireless communication.

### II. Example Operation of a Multifrequency Ultrasound System

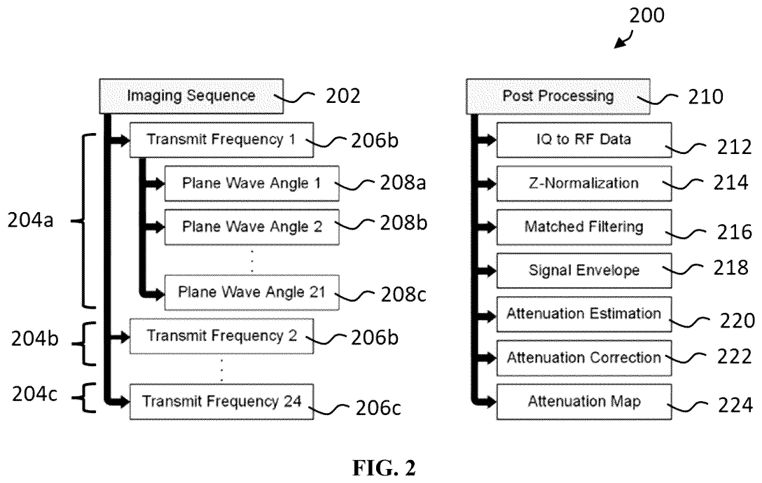

FIG. 2 illustrates an example method 200 of operating a multifrequency ultrasound system, such as the system 100 of FIG. 1. An imaging sequence 202 is first performed. The imaging sequence 202 includes a number of measurement steps 204a-c. Each step 204a-c is performed at a corresponding ultrasound transmission frequency 206a-c. At each frequency 206a-c, the ultrasound signal may be transmitted at a number of plane wave angles 208a-c to obtain data from a region-of-interest in the specimen. In ultrasound imaging, a plane wave angle refers to the angle at which a plane wave, which is a type of ultrasound beam where all wavefronts are parallel, is transmitted from the transducer towards the specimen being imaged. The use of different angles allows more comprehensive imaging by capturing data from various directions. The imaging sequence 202 may be fully automated.

Post processing 210 is then performed using the data obtained from the imaging sequence 202. In the example method 200, the data obtained from the imaging sequence 202 is provided to the postprocessing unit and stored as IQ data to perform post processing 210. At operation 212, the IQ data is converted to RF data. The IQ-to-RF conversion reconstructs the radiofrequency signal from its in-phase (I) and quadrature (Q) components, which represent the real and imaginary parts of the complex baseband signal. Following IQ to RF reconstruction, z-normalization is applied to the RF data at operation 214 to compensate for intensity changes between the different transmit frequencies, for example, caused by inefficiencies in the transducer output when deviating from the default resonant frequency. Z-normalization involves computing, for each transmission frequency, the mean (u) and standard deviation (c) of the RF signal amplitudes across the image, and then transforming each amplitude value x to a normalized value z=(x−μ)/σ. This statistical normalization ensures that intensity comparisons across different transmission frequencies reflect true tissue attenuation differences rather than transducer response variations. The use of z-normalization helps to limit the impact of these intensity changes and is particularly important for accurate attenuation coefficient estimation.

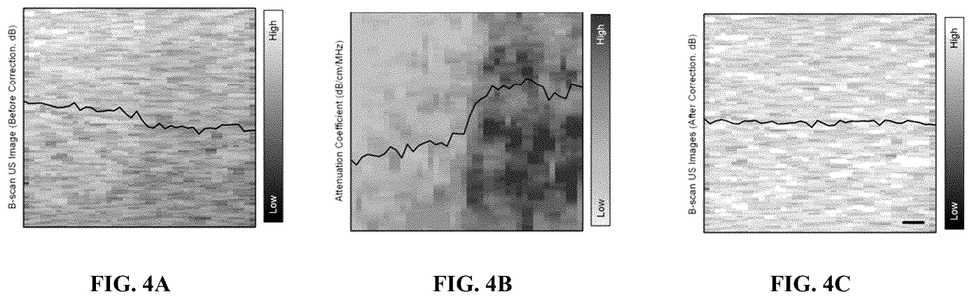

At operation 216, matched filtering is performed, for example, using frequency-shifted Gaussian filters for each discrete transmission frequency in the acquisition sequence (steps 204a-c). The center frequency and bandwidth for each filter may be matched to the transmission signal from the step 204a-c in the imaging sequence 202. A Gaussian filter with center frequency fc and bandwidth B has a frequency response that follows a Gaussian distribution centered at fc, effectively passing signal components near the transmission frequency while attenuating off-frequency noise and interference. By applying a bank of matched Gaussian filters corresponding to each transmission frequency, the system isolates the backscattered signal content at each discrete frequency for subsequent attenuation analysis. At operation 218, raw (e.g., B-scan) ultrasound images are produced, for example, using the signal envelope of the RF data. An example raw ultrasound image is shown in FIG. 4A (described further with respect to the Examples below). The signal envelope refers to a curve that outlines the maximum amplitude variations of the radio frequency signal over time, providing a quantitative representation of the shape of the signal's amplitude fluctuations, without considering the rapid carrier wave oscillations at the high RF frequency. The signal envelope may be calculated using the Hilbert transformation, which computes the analytic signal representation of the RF data, from which the instantaneous amplitude (envelope) is obtained as the magnitude of the complex analytic signal. The envelope values are then converted to a logarithmic scale (e.g., decibels) for display and subsequent attenuation analysis.

At operation 220, attenuation is estimated. Attenuation is primarily dependent on a spatial attenuation coefficient (α_{i,j}), ultrasound transmission frequency (f_{c}), and image depth (zᵢ). Attenuation estimations are performed using the raw (B-scan) ultrasound image data. FIG. 3 illustrates an example method for attenuation estimation at operation 220. The disclosed attenuation estimation algorithm exploits the physical relationship between ultrasound signal attenuation and transmission frequency, wherein higher frequency ultrasound signals experience greater attenuation at a given tissue depth. By acquiring ultrasound data at multiple discrete frequencies and analyzing the frequency-dependent intensity changes at each pixel location, the local attenuation coefficient can be determined without requiring a reference phantom or assumptions about global tissue homogeneity. To determine the attenuation coefficient for each pixel individually, a least-squares regression may be computed for the ultrasound image intensity (e.g., in units of decibels, dB) versus the transmit frequency (MHz). Specifically, for each pixel location (i, j), the intensity values Ii,j(f1), Ii,j(f2), . . . . Ii,j(fN) obtained at N different transmission frequencies f1, f2, . . . fN are fitted to a linear model. The slope of this line (see linear fits in FIG. 3) represents the frequency-dependent attenuation at that pixel location. This slope is then divided by the depth zi (e.g., in units of centimeters, cm) to obtain the estimated attenuation coefficient αi,j in units of dB/cm/MHz. This pixel-by-pixel approach produces a high-resolution spatial map of attenuation coefficients, enabling localized correction that accounts for tissue heterogeneity.

Returning to FIG. 2, at operation 222, attenuation correction is performed on the RF data. The attenuation-corrected RF data (RF_{i,f}^{AC}) may be determined using a correction factor with an exponential term that includes the determined attenuation coefficient (α_{i,j}) for the corresponding position of the RF data, the frequency (f_{c}) at which the data was obtained, and the image depth (zi). The correction algorithm applies a multiplicative correction factor to each RF data sample to compensate for the signal loss that occurred during propagation through the tissue. Specifically, the attenuation-corrected RF data is computed according to:

$${RF}_{i , j}^{AC} = {RF}_{i , j} \times {10}^{\frac{- {α}_{i , j} {f}_{c} {z}_{i}}{20}} \qquad ( 1 )$$

where RFi,j is the original radiofrequency data at pixel location (i, j), αi,j is the estimated attenuation coefficient at that location, fc is the transmission frequency, and zi is the depth. The factor of 20 in the denominator of the exponent accounts for the conversion between amplitude and power in decibel units (since attenuation coefficients are typically expressed in dB/cm/MHz). This correction effectively reverses the attenuation that occurred during signal propagation, amplifying deeper signals that experienced greater attenuation while leaving shallower signals relatively unchanged. Because the correction is applied on a pixel-by-pixel basis using locally-estimated attenuation coefficients, heterogeneous tissues with varying attenuation properties are corrected more accurately than with conventional global correction approaches.
The attenuation-corrected RF data is used to generate an attenuation-corrected ultrasound image in a similar manner to the generation of the raw image data generated at operation 218 (e.g., using the signal envelope of the attenuation-corrected RF data). An example attenuation-corrected ultrasound image is shown in FIG. 4C (described further with respect to the Examples below). The attenuation-corrected ultrasound image may be color coded, for example, such that lower frequency backscattered ultrasound signal components are assigned to a certain color channel (e.g., red) and higher frequency signals are assigned a different color (e.g., blue). Color coding may facilitate detection of relative scatterer size for tissue characterization. Colors may be selected in accord with human perception of visual light to improve interpretation and visualization of the attenuation-corrected ultrasound image.

At operation 224, an attenuation map is generated. The attenuation map includes data which may represent image pixels representing the value of the attenuation coefficient determined for each pixel of the raw image data. An example attenuation map is shown in FIG. 4B (described further with respect to the Examples below). The attenuation map may be presented with intensity-scaled values for each pixel or using a color code. For example, a maximum attenuation coefficient and a minimum attenuation coefficient in the attenuation map may be determined. The attenuation map may be scaled based on the maximum and minimum attenuation coefficients (e.g., ranging from dark to light, blue to red, or the like). Colors may be selected in accord with human perception of visual light to improve interpretation and visualization of the attenuation map.

### EXAMPLES

The following examples are included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments, which are disclosed and still obtain a like or similar result without departing from the spirit and scope of the invention. The systems and methods of this disclosure may provide tools for noninvasive imaging.

### Example Implementation

In an example implementation of the systems and methods of this disclosure, a programmable research scanner (Vantage NXT 256, Verasonics Inc) equipped with a 128-element L11-5v linear array transducer was used in a plane wave imaging mode. Backscattered radiofrequency data at 24 different transmit frequencies that span the range of the transducer bandwidth were sequentially collected. Each acquisition involved 21 angled planes waves in the ±18° range to improve the signal-to-noise ratio and resolution before coherent summation. The data acquisition process took approximately 3 seconds for all 24 transmissions and reconstructions. Local attenuation coefficients were estimated from the series of multifrequency ultrasound images based on the relationship between ultrasound image intensity, transmit frequency, and depth at each spatial (pixel) location (see FIG. 3). This approach produced high resolution maps or images of the ultrasound attenuation coefficient at high resolution.

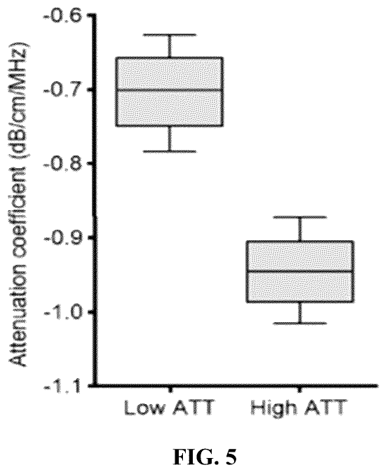

Preliminary multifrequency ultrasound imaging tests were conducted using an ultrasound phantom (Model 040GSE, CIRS Inc) having two different homogeneous material regions with known attenuation coefficients of −0.70 and −0.95 dB/cm/MHz. Multifrequency ultrasound images were acquired from both sections, and ultrasound attenuation images were summarized as mean plus/minus standard deviation (FIG. 5). Attenuation estimates were determined for a defined region-of-interest (ROI). A sliding window was used to average noise in the axial direction. The window size was arbitrarily defined as 41 rows, or approximately 2 mm. The window was incremented one row at a time to produce the final attenuation map of FIG. 4B.

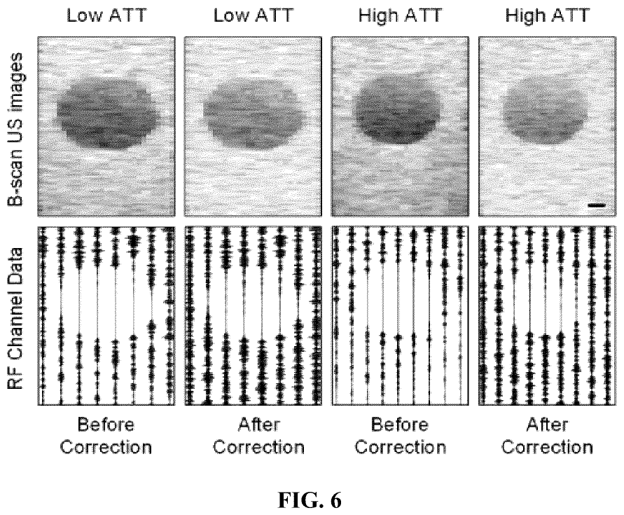

The attenuation was controlled using an additive such that there is no measurable backscatter difference between the two sides of the phantom. Ultrasound imaging was performed in three areas: the middle region between the two sides, the hypoechoic cyst in the low attenuation (Low ATT) side, and the anechoic cyst in the high attenuation (High ATT) side (see FIG. 6). Imaging down the center of the phantom was performed to assess the ability of the attenuation estimation to correct both phantom sides in a singular ROI. These ROIs were placed in deep regions of the image (>2.5 cm) to ensure that the relationship between B-scan US image intensity and depth is linear. As demonstrated by the results reported below (see FIG. 5), the measured attenuation coefficients largely matched the phantom's specifications.

The phantom's cyst targets were imaged to analyze the effect of having a heterogenous target in the ROI, and to ensure that the cyst structure was not lost after correction (see FIG. 6). During this imaging process, the Vantage research scanner (Vantage NXT 256, Verasonics Inc) was used to generate 24 frequency-shifted narrowband ultrasound pulses spaced along the bandwidth of the transducer from 4.71 to 10.4 MHz. Transmission frequencies were selected to have relatively equal spacing throughout the bandwidth of the transducer. Received data was filtered using a set of Gaussian filters with center frequencies corresponding to the transmit frequency. Increasing the number of pulse cycles from 1 to 3 had the effect of narrowing the frequency bandwidth of the return signal, which provided improved frequency sensitivity and reduced overlap between each transmission. Traditional imaging uses a single pulse cycle duration with a center frequency of 7.6 MHz. However, to achieve improved frequency sensitivity and reduced overlap between each transmission frequency, three pulse cycles were performed. In the frequency domain, longer pulse cycles have the effect of narrowing the received spectrum. The filtered radiofrequency data was converted to B-scan US images using the Hilbert transformation along with logarithmic scaling. After the 24th frequency, a standard 1 cycle pulse at the default resonant frequency was also acquired. This produces 24 B-scan US images from which the 2D attenuation map is generated pixel-by-pixel using the linear relationship between logarithmic image intensity and frequency, as described above (see also FIG. 3).

B-scan US images and the corresponding attenuation map are summarized in FIGS. 4A-C, when imaged down the center of the phantom. To estimate the effectiveness of the attenuation correction, line plots were overlaid on the images of FIGS. 4A and 4C corresponding to depth-averaged B-scan US image intensity and attenuation coefficients. The corrected image (FIG. 4C) displayed relatively uniform intensity levels laterally and axially across both sides when compared to the raw image (FIG. 4A). In the attenuation map (FIG. 4B), there is a high degree of sensitivity when estimating along the boundary of the two sides.

FIG. 5 shows a box chart of the attenuation coefficients determined for the different materials in the phantom to evaluate the accuracy of the attenuation estimation relative to the known phantom specifications. The ROIs were placed in the same position of the imaging plane for both phantom sides. For the low and high attenuation estimations, coefficients of −0.70±0.06 and −0.95±0.05 dB/cm/MHz were determined, respectively. When compared to the known attenuation coefficient, a maximum error of approximately 7.9 and 5.2% was observed, respectively. It is believed that diffraction, beamforming, and time gain compensation (TGC) effects were the primary cause of deviations from the known values. Normalizing the transmit power between each frequency may be performed to improve accuracy.

Spatial correction was demonstrated using images 602, 606, 610, 614 of the hypoechoic cyst targets in the phantom, as shown in FIG. 6. The primary goal of obtaining these attenuation-corrected images was to confirm that the cyst structure was still observable after correction. RF lines 604, 608, 612, 616 for representative channels demonstrate the impact of attenuation correction. Uniform RF lines 604, 608, 612, 616 were obtained for the background region after correction, and the structure of the cyst was largely retained. This demonstrates the ability of the spatial attenuation estimation to function on heterogeneous images for use in the assessment of disease pathology. For example, breast tissue carcinoma and liver steatosis exhibit higher degrees of attenuation when compared to healthy tissue (F. T. D'Astous and F. S. Foster, Ultrasound Med. Biol., vol. 12, no. 10, pp. 795-808, 1986). Correcting this increased attenuation may improve the accuracy of quantitative ultrasound techniques. Furthermore, tracking changes in attenuation coefficients may be a promising approach to non-invasive determination of disease pathology.

All of the systems and methods disclosed and claimed herein can be made and executed without undue experimentation in light of the present disclosure. While the systems and methods of this invention have been described in terms of preferred embodiments, it will be apparent to those of skill in the art that variations may be applied to the systems and in the steps or in the sequence of steps of the method described herein without departing from the concept, spirit, and scope of the invention. All such variations and modifications apparent to those skilled in the art are deemed to be within the spirit, scope, and concept of the invention as defined by the appended claims.

What is claimed is:

1. A system, comprising:
an ultrasound transducer configured to:
iteratively transmit an ultrasound signal into a specimen at a plurality of frequencies;
detect backscattered ultrasound at each of the plurality of frequencies; and
generate radiofrequency (RF) data for each of the plurality of frequencies; and
a postprocessing unit comprising a processor configured to:
receive at least a portion of the RF data;
transform the received RF data into a raw image comprising first pixels, each first pixel comprising an intensity value;
for each first pixel of the raw image, determine an attenuation coefficient based on at least one of: the intensity value of the first pixel, a transmission frequency at which the intensity value was obtained, and a depth within the specimen at which the intensity value was obtained;
generate attenuation-corrected RF data using the determined attenuation coefficients;
transform the attenuation-corrected RF data into an attenuation-corrected ultrasound image comprising second pixels, each second pixel comprising an attenuation-corrected intensity value determined based at least in part on the determined attenuation coefficients; and
provide the attenuation-corrected image for display on a display device.

2. The system of claim 1, wherein the processor of the postprocessing unit is further configured to determine the attenuation coefficient based on each of the intensity value of the first pixel, the transmission frequency at which the intensity value was obtained, and the depth within the specimen at which the intensity value was obtained.

3. The system of claim 1, wherein the processor of the postprocessing unit is further configured to, for each frequency of the plurality of frequencies, generate the attenuation-corrected RF data using a correction factor with an exponential term that comprises an attenuation coefficient of the determined attenuation coefficients and the frequency.

4. The system of claim 1, wherein the processor of the postprocessing unit is further configured to normalize the RF data to compensate for intensity changes of the backscattered ultrasound at the plurality of frequencies of the ultrasound signal transmitted by the ultrasound transducer.

5. The system of claim 1, wherein the processor of the postprocessing unit is further configured to filter the RF data.

6. The system of claim 1, wherein the processor of the postprocessing unit is further configured to:
generate an attenuation map comprising a plurality of third pixels, each third pixel comprising the attenuation coefficient determined for each first pixel of the raw image; and
provide the attenuation map for display on the display device.

7. The system of claim 1, wherein the processor of the postprocessing unit is further configured to:
receive a broadband ultrasound image of the specimen;
adjust the broadband ultrasound image using the attenuation coefficients, thereby generating an enhanced broadband ultrasound image.

8. A method, comprising:
iteratively transmitting, by an ultrasound transducer, an ultrasound signal into a specimen at a plurality of frequencies;
detecting backscattered ultrasound at each of the plurality of frequencies;
generating radiofrequency (RF) data for each of the plurality of frequencies;
transforming at least a portion of the RF data into a raw image comprising first pixels, each first pixel comprising an intensity value;
for each first pixel of the raw image, determining an attenuation coefficient based on at least one of: the intensity value of the first pixel, a transmission frequency at which the intensity value was obtained, and a depth within the specimen at which the intensity value was obtained;
generating attenuation-corrected RF data using the determined attenuation coefficients; transforming the attenuation-corrected RF data into an attenuation-corrected ultrasound image comprising second pixels, each second pixel comprising an attenuation-corrected intensity value determined based at least in part on the determined attenuation coefficients; and
providing the attenuation-corrected image for display on a display device.

9. The method of claim 8, further comprising determining the attenuation coefficient based on each of the intensity value of the first pixel, the transmission frequency at which the intensity value was obtained, and the depth within the specimen at which the intensity value was obtained.

10. The method of claim 8, further comprising, for each frequency of the plurality of frequencies, generating the attenuation-corrected RF data using a correction factor with an exponential term that comprises an attenuation coefficient of the determined attenuation coefficients and the frequency.

11. The method of claim 8, further comprising normalizing the RF data to compensate for intensity changes of the backscattered ultrasound at the plurality of frequencies of the ultrasound signal transmitted by the ultrasound transducer.

12. The method of claim 8, further comprising filtering the RF data.

13. The method of claim 8, further comprising:
generating an attenuation map comprising a plurality of third pixels, each third pixel comprising the attenuation coefficient determined for each first pixel of the raw image; and
providing the attenuation map for display on the display device.

14. The method of claim 13, further comprising:
receiving a broadband ultrasound image of the specimen;
adjusting the broadband ultrasound image using the attenuation coefficients, thereby generating an enhanced broadband ultrasound image.

15. A system, comprising a processor configured to:
receive radiofrequency (RF) data generated by an ultrasound transducer at a plurality of frequencies, the RF data associated with a specimen;
transform the received RF data into a raw image comprising first pixels, each first pixel comprising an intensity value;
for each first pixel of the raw image, determine an attenuation coefficient based on at least one of: the intensity value of the first pixel, a transmission frequency at which the intensity value was obtained, and a depth within the specimen at which the intensity value was obtained;
generate attenuation-corrected RF data using the determined attenuation coefficients;
transform the attenuation-corrected RF data into an attenuation-corrected ultrasound image comprising second pixels, each second pixel comprising an attenuation-corrected intensity value determined based at least in part on the determined attenuation coefficients; and
provide the attenuation-corrected image for display on a display device.

16. The system of claim 15, wherein the processor is further configured to determine the attenuation coefficient based on each of the intensity value of the first pixel, the transmission frequency at which the intensity value was obtained, and the depth within the specimen at which the intensity value was obtained.

17. The system of claim 15, wherein the processor is further configured to, for each frequency of the plurality of frequencies, generate the attenuation-corrected RF data using a correction factor with an exponential term that comprises an attenuation coefficient of the determined attenuation coefficients and the frequency.

18. The system of claim 15, wherein the processor is further configured to normalize the RF data to compensate for intensity changes of backscattered ultrasound detected by the ultrasound transducer at the plurality of frequencies of the ultrasound signal transmitted by the transducer.

19. The system of claim 15, wherein the processor is further configured to filter the RF data.

20. The system of claim 15, wherein the processor is further configured to:
generate an attenuation map comprising a plurality of third pixels, each third pixel comprising the attenuation coefficient determined for each first pixel of the raw image; and
provide the attenuation map for display on the display device.
